(19) United States
(12) Patent Application Publication
(10) Pub. No.: US 2002/0009474 A1
(43) Pub. Date: Jan. 24, 2002

(54) METHOD AND COMPOSITION FOR INCREASING CALCIUM UPTAKE

(76) Inventors: PRASAD S. ADUSUMILLI, WARREN, NJ (US); KENNETH G. MANDEL, PARSIPPANY, NJ (US); HARESH L. MIRCHANDANI, MORRIS PLAINS, NJ (US)

Correspondence Address: KNOBBE, MARTENS, OLSON & BEAR, LLP, 620 NEWPORT CENTER DRIVE, 6TH FLOOR, NEWPORT BEACH, CA, 92660, US

(21) Appl. No.: 09/287,495

(22) Filed: Apr. 6, 1999

Related U.S. Application Data
(63) Continuation-in-part of application No. 08/522,337, filed on Nov. 7, 1995, now abandoned.
Application No. 08/522,337 is a continuation-in-part of application No. 08/355,537, filed on Dec. 14, 1994, now abandoned.
Application No. 08/355,537 is a continuation-in-part of application No. 08/242,753, filed on May 13, 1994, now abandoned.

(30) Foreign Application Priority Data
May 12, 1995 (US) US95/06239

(51) Int. Cl. A01N 25/00; A61K 33/14; A01N 59/08

(52) U.S. Cl. 424/405; 424/678

(57) ABSTRACT

This invention relates to a hydrophilic matrix containing a pharmaceutically acceptable calcium source for use as a calcium supplement for mammals.

### RELATED INFORMATION

This application is a continuation in part application of U.S. Ser. No. 08/522,377 (allowed) filed Nov. 12, 1996 which is the §371 national stage entry of PCT/US95/06239, filed May 12, 1995 which is a continuation in part application of U.S. Ser. No. 08/355,537, filed Nov. 7, 1994 (abandoned) which is a continuation in part application of U.S. Ser. No. 08/242,753, filed May 13, 1994 (abandoned).

### FIELD OF THE INVENTION

This invention relates to a hydrophilic matrix containing a pharmaceutically acceptable calcium source for use as a calcium supplement for mammals.

### BACKGROUND OF THE INVENTION

Calcium supplements are widely used for the treatment of osteoporosis. The bioavailability of these preparations are relatively low. This problem has been noted by a number of medical professionals recently as the issue of osteoporosis in an aging population is becoming a well publicized issue (F-D-C-Reports—“The Tan Sheet”, Mar. 14, 1994). Not all calcium supplements are the same. Bioavailability appears to differ among and between sources of calcium. It often is influenced by manufacturing processes. Solubility in vitro is not necessarily correlated with bioavailability. And even though the same total amount of calcium is ingested, more calcium may be absorbed if the supplement is taken in multiple small doses, especially when taken with food, that when taken in just a few large doses. Other nutritional depravations, such as inadequate vitamin D intake, may influence calcium absorption. What ever the reason, calcium absorption from supplements can be quite variable from preparation to preparation and is not a particularly efficient process vis-a-vis the currently available oral supplements.

Adequate calcium intake whether from food or supplements is important in both preventive and treatment regimens for osteoporosis and osteomalacia. The use of calcium supplements has increased dramatically in recent years. Some recent evidence indicates that calcium intake may be associated with a reduced risk of colon cancer and may have a blood-pressure lowering effect. If these initial results are verified it will likely further increase the use of calcium supplements, making it more important that optimal dosing regimens be developed to minimize toxicity and maximize their efficacy [J. Blanchard and J M Aeschlimann. Calcium Absorption in Man: Some Dosing Recommendations. J. Pharmacokinetics and Biopharmaceutics, 17(6), 1989, 631-644]. Concern has been expressed that the bioavailability of calcium from many calcium carbonate supplements is low. For most commercially available products, calcium absorption in adults commonly averages 25-35% of the available calcium in the dosage form. The low bioavailability could be attributed to either an incomplete drug release or to a too short residence time of the pharmaceutical dosage form in the absorption section of the GI Tract (H. M. Ingani, et al. Conception and in vivo investigation of peroral sustained release floating dosage forms with enhanced gastrointestinal transit. Int. J. Pharm., 35 (1987) 157-164). Therefore, design of better delivery systems seem to be necessary. Some researchers have shown that the absorption of calcium involves a saturable (active) and a nonsaturable (passive) component. The combination of the acidic pH and calcium binding protein in the duodenum and upper jejunum makes the absorption of calcium much greater in the duodenojejunal section [Lindsay H. Allen. Calcium bioavailability and absorption: a review. Am J Clin Nutr., 1982 ; 35;783-808]. The work of several other investigators indicate that an inverse relationship exists between calcium intake and absorption efficiency. The division of the daily dose into equal increments taken at equally spaced interval over the course of the day is recommended as a useful procedure for increasing the absorption efficiency and efficacy. In addition, it is reported that single unit systems can be retained in the stomach for long periods (10 hours and longer) if administered after a heavy meal [R. Malagelada et al. The stomach, but not small bowel, discriminates between solid and liquids in man. Gastroenterology, 84 (1983) 1237].

Previous studies aimed at increasing calcium uptake in the intestine have mainly concentrated on increasing the dissolution rate and increasing the solubility of the calcium source. Based on this information, a controlled release calcium oral dosage form with increased gastric retention time is an appropriate vehicle to increase the bioavailability of calcium.

### SUMMARY OF THE INVENTION

The present invention relates to a method of increasing calcium absorption in a mammal in need thereof, which method comprises administering to said mammal, a pharmaceutically acceptable oral preparation containing a source of calcium ion in solid form encapsulated with a hydrophilic matrix polymer, which is a rafting agent, whereby the polymer increases the residence time in the GI tract of the calcium supplement allowing increased absorption of the calcium..

The present invention also relates to a pharmaceutically acceptable oral calcium supplement preparation comprising a source of calcium ion in solid dosage form wherein the calcium source is entrapped with a hydrophilic matrix polymer, which polymer is a rafting agent, which polymer increases the residence time in the GI tract of the calcium supplement. In yet a further aspect, this invention comprises a method for increasing calcium absorption in a mammal which method comprises administering to a mammal in need of a calcium supplement a pharmaceutically acceptable solid dosage form containing a source of calcium entrapped in a hydrophilic matrix which results in increased retention of the matrix material in the stomach.

### BRIEF DESCRIPTION OF THE FIGURES

FIG. 1 shows graphic data of calcium released from HPMC at three concentration levels (viscosity grade: 100cps).

FIG. 2 shows graphic data of calcium released from HPMC at three concentration levels (viscosity grade 2050cps).

FIG. 3 shows graphic data of calcium released from HPMC at three concentration levels (viscosity grade: 4000cps).

FIG. 4 shows graphic data of calcium released from HPMC at three viscosity grades (5% HPMC).

FIG. 5 shows graphic data of calcium released from HPMC at three viscosity grades (12.5% HPMC).

FIG. 6 shows graphic data of calcium released from HPMC at three viscosity grades (20% HPMC).

### DETAILED DESCRIPTION OF THE INVENTION

To achieve the goal of the present invention, a controlled release calcium oral dosage form with increased gastric retention time to increase the bioavailability of calcium, a hydrophilic matrix system is used to encapsulate a source of calcium ion, such as calcium carbonate. The matrix material utilized herein was different grades of hydroxypropylmethylcellulose (HPMC). The grades of HPMC represent a variety of polymers with different molecular weights. Methocel K100LV and K4M were selected as representative polymers. It is reported that these polymers demonstrate floating and swelling behavior [V.S. Gerogiannis, et al., Floating and swelling characteristics of various excipients used in controlled release technology, Drug Dev. Ind. Pharm., 19(9),1061-1081 (1993)].

Similarly, it is well known that alginic acids and their salts precipitate in gastric acid to form viscous gels. Including bicarbonate in a formulation along with these alginic acids and their salts allows these gels to float on the surface of gastric contents. This property enables the floating matrix to reside in the stomach for prolonged periods of time. If other agents such as drug molecules or mineral salts are included in the formulation, they can become entrapped within the gel matrix, and their release and subsequent absorption can be controlled [G. L. Beckloff, et al., Objective evaluation of an antacid with unusual properties, J. Clin. Pharmacol. New Drugs, 12(1),11-21 (1972); H. A. May, et al., Monitoring radiolabeled antacid preparations in the stomach, Int. J. Pharm., 19,169-176 (1984); N. Washington and C. G. Wilson, Antacids: physiology versus pharmaceutics, Int. J. Pharm., 28,249-260 (1986); N. M. Davies, et al., A comparison of the gastric retention of alginate containing tablet formulations with and without the inclusion of excipient calcium ions, Int. J. Pharm., 105,97-101 (1994); J. Vatier, et al., A computerized artificial stomach model to assess sodium alginate-induced pH gradient, Int. J. Pharm., 163,225-229 (1998)].

In its broadest aspect, this invention provides a means for increasing the gastric uptake of calcium in a mammal from a calcium supplement by the expedient of forming a matrix around the calcium material with a hydrophilic polymer and confecting that material into an oral dosage form. It has been found that the hydrophilic material forming matrix swells in water and as a result prolongs the gastric residence time and/or prevents erratic gastric emptying during the digestive phase. Consequently the calcium source enjoys a longer residence time in the upper intestine thereby enhancing the amount of calcium which can be absorbed.

These formulations and processes are concerned only with solid or viscous, oral dosage forms. Tablets or chewable troches or lozenges are of most interest herein. And this technology will have the most successful application in these types of preparations. As for the makeup of the product, it will comprise a calcium source entrapped in a matrix of hydrophilic polymer, this complex being formulated into a tablet, troche, lozenge or the like using conventional methods and conventional excipients.

The calcium source which can be used in this invention is any pharmaceutically acceptable calcium salt or chelated calcium product. The following exemplary calcium products have been used in supplements: calcium carbonate, calcium phosphate, calcium citrate, calcium gluconate, calcium oxalate, and glycine calcium. Other forms of calcium can also be used, provided they are acceptable for human use.

Hydrophilic matrix forming polymers are used to entrap the calcium source. These polymers will be non-toxic, that is safe for human consumption when administered orally. Useful examples of such polymers are the celluloses, particularly hydroxypropyl methyl cellulose (HPMC), as well as alginic acids and their salts. These polymers, and the group of polymers of like nature, provide two benefits: i) the matrix they form with the calcium source effects a sustained release preparation, and ii) when exposed to aqueous media, especially the acid environment of the stomach, the polymers demonstrate a swelling and/or floating behavior. Both characteristics contribute to a longer residence time in the GI tract, primarily in the stomach. Longer term exposure contributes to more calcium being absorbed by the gut.

In a preferred embodiment of this invention the hydrophilix matrix forming polymer is an alginic acids or salt thereof, or a mixtures thereof, alone or in combination with a second hydrophilix matrix forming polymer such as the celluloses, preferably HPMC as described herein.

Tablets, troches, lozenges and the like can be prepared using standard practices and procedures. Excipients can be selected from any of the known, pharmaceutically acceptable materials currently used or known for making said tablets, etc. See for example Remington's Pharmaceutical Sciences, A. R Gennaro Ed., 18th Edition, Mack Publishing Co, Easton, Pa., USA (1990) and similar reference works.

Other components can be added into these preparations. For example an antacid can be added to neutralize stomach acid. Another example is various formulations of alginic acid or their salts, together with these antacids. Such products are often marketed under the brand name Gaviscon®. Another suitable component may be a drug associated with bone mineral resorption or one which enhances calcium uptake, for example, which might be also be added into the formulation. Flavoring agents, colors-imparting agents, preservatives, etc., can be included as well and are within the skilled artisans knowledge.

The following examples are set out as a means of illustrating the invention. They are not intended to limit the invention and should not be interpreted to do so. Reference is made to the claims for what is reserved to the inventors hereunder.

### EXAMPLES

### Example 1

Preparation of Tablets

A dry blend consisting of calcium carbonate (2 kg) and Methocel E5 (100 mg, 5% of calcium carbonate) was mixed in a bowl mixer (Hobart Mixer) for 5 min. Approximately 360 ml of distilled water was added gradually to form a wet granulation, which was then dried overnight in an oven set at 40° C. The dry granules were screened through a 20 mesh sieve and stored as stock granules. This granulation was then mixed with different percentages of Methocel K100LV and/or K4M as per Table I below for 20 min. Finally 2.5% of stearic acid and 0.5% of supernat were added and mixed for another 5 min. in a V-blender. The blend so obtained was compressed into tablets using a Stokes single punch press (Key Industrials Inc., Englishtown, N.J.) to make the tablets. The average hardness for all formulations were adjusted to 14.6-17.2 scu and the weight variation of all nine formulations were within 10% of the target weights. The elemental calcium in each tablet is approximately 250 mg.

Experimental Design

Nine formulations were generated using a 3² full factorial design. Using different levels and grades of HPMC, the viscosity and concentration of HPMC in tablets were optimized to achieve the desired in vitro dissolution profile. Independent variables used in the design were viscosity of the polymeric system and concentration of the polymer system in the dosage form. Viscosity and concentration were allowed to vary from 100 cps (−1 ) to 4000 cps ( 1) and 5% HPMC (−1) to 20% ( 1), respectively. Percent of calcium dissolved at 1, 2, 3, 4, 6, 8 and 10 hours were used as the dependent variables. The experimental design is given below in Table 1.

TABLE 1

| | |
|---|---|
| Exp. | Controlled Factors |

| Number | Viscosity | Concentration |
|---|---|---|
| 1 | −1 | −1 |
| 2 | −1 | 0 |
| 3 | −1 | 1 |
| 4 | 0 | −1 |
| 5 | 0 | 0 |
| 6 | 0 | 1 |
| 7 | 1 | −1 |
| 8 | 1 | 0 |
| 9 | 1 | 1 |

The viscosity of Methocel K100LV is 100 cps while Methocel K4M is 4000 cps. Eighth root equation (METHOCEL Cellulose Ethers Technical Handbook, Dow Chemical U.S.A. Midland, Mich.) was employed to calculate the correct blend needed to yield the intermediate viscosity.

Dissolution of the nine formulations was measured using USP apparatus 1 (Vanderkamp 600 dissolution apparatus, Van-Kel Industries, Edison, N.J.) at 100 rpm in 900 ml of 0.1 N HCl kept at 37° C. and aliquots (3 ml) of the dissolution medium was withdrawn from each vessel at 1 hour, 2 hours, 3 hours, 4 hours, 6 hours, 8 hours and 10 hours. All samples were filtered through 0.45 μm cellulose acetate membranes, diluted with distilled water and analyzed for dissolved calcium by Atomic Absorption Spectrophotometry (Varian Techtron Pty. Ltd. Springvale, Australia). Percentage of dissolved calcium was calculated by dividing the measured calcium content of a particular sample by the calcium weight of the tablet.

An inverse relationship was found to exist between concentration of the HPMC matrix and calcium release rate. Results of the in vitro dissolution tests are shown in Table 2.

TABLE 2

| | Exp. | Exp. | Exp. | Exp. | Exp. | Exp. | Exp. | Exp. | Exp. |
|---|---|---|---|---|---|---|---|---|---|
| | 1 | 2 | 3 | 4 | 5 | 6 | 7 | 8 | 9 |
| Wgt | 809.3 | 865.3 | 899.8 | 817.4 | 867.2 | 907.3 | 801.2 | 850.8 | 911.0 |
| (mg)* | | | | | | | | | |
| Hard- | 16.5 | 16.1 | 17.2 | 16.5 | 16.5 | 14.6 | 16.4 | 16.8 | 15.8 |
| ness* | | | | | | | | | |
| (scu) | | | | | | | | | |

% of Ca Release**

| | | | | | | | | | |
|---|---|---|---|---|---|---|---|---|---|
| 1 h | 40.1 | 20.8 | 20.9 | 24.8 | 18.5 | 12.1 | 21.3 | 14.2 | 10.9 |
| 2 h | 63.4 | 43.9 | 38.2 | 36.4 | 32.9 | 20.4 | 32.6 | 23.0 | 17.3 |
| 3 h | 71.4 | 67.4 | 54.9 | 53.2 | 45.8 | 28.7 | 45.3 | 33.1 | 25.0 |
| 4 h | 81.5 | 75.5 | 67.5 | 67.8 | 58.1 | 38.5 | 58 | 40.6 | 29.9 |
| 6 h | 92.7 | 91.8 | 76.6 | 86.7 | 76.4 | 58.8 | 82.6 | 71.1 | 40.7 |
| 8 h | 95.2 | 95.5 | 90.5 | 93.8 | 90.5 | 82.6 | 91.3 | 82.6 | 52.7 |
| 10 h | 97.3 | 96.4 | 93.3 | 97.0 | 93.5 | 87.9 | 95.4 | 91.4 | 63.0 |

*The weight and hardness are the average of ten tablets.
**The percentage of calcium dissolved is the mean of four tablets.

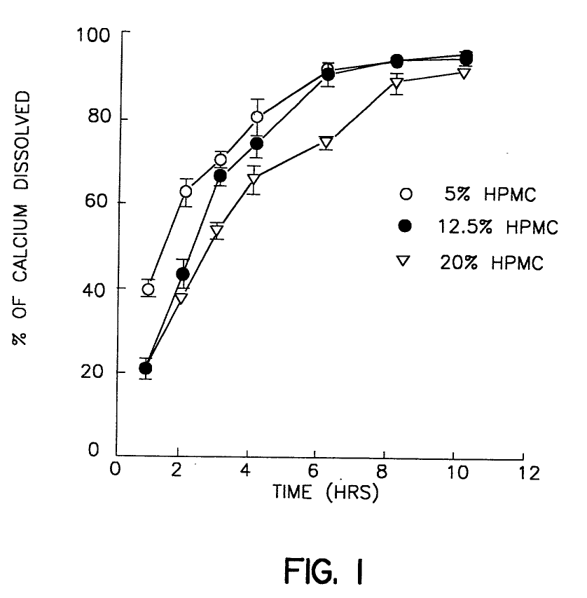

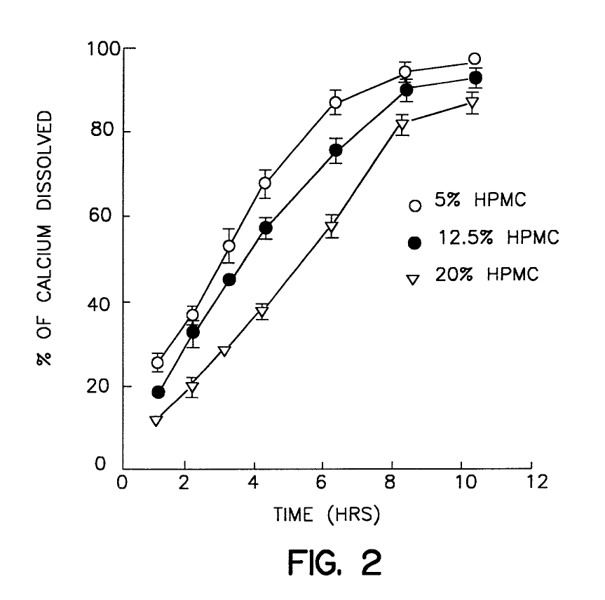

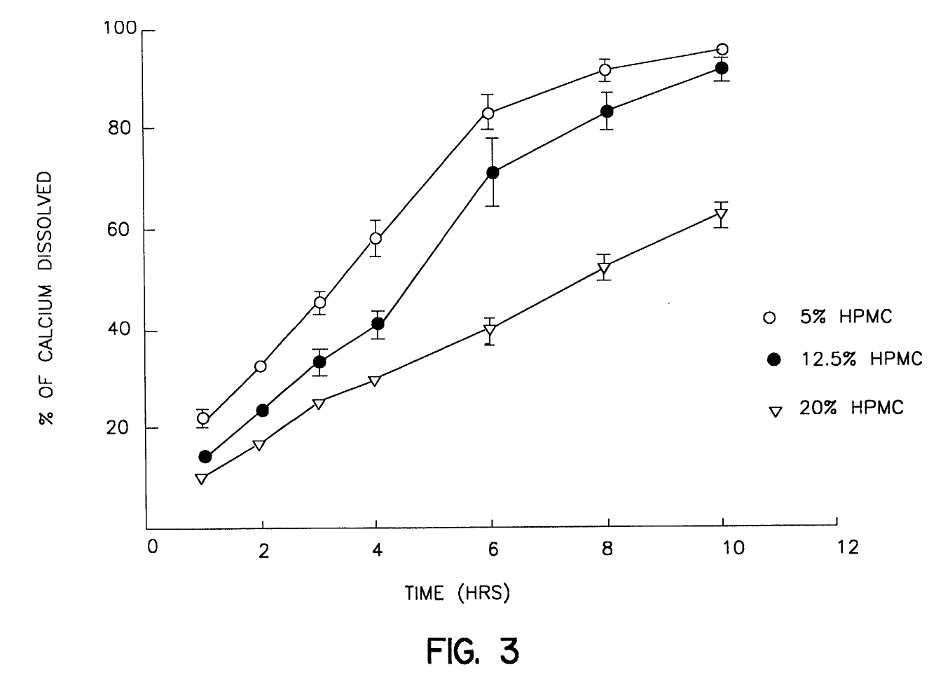

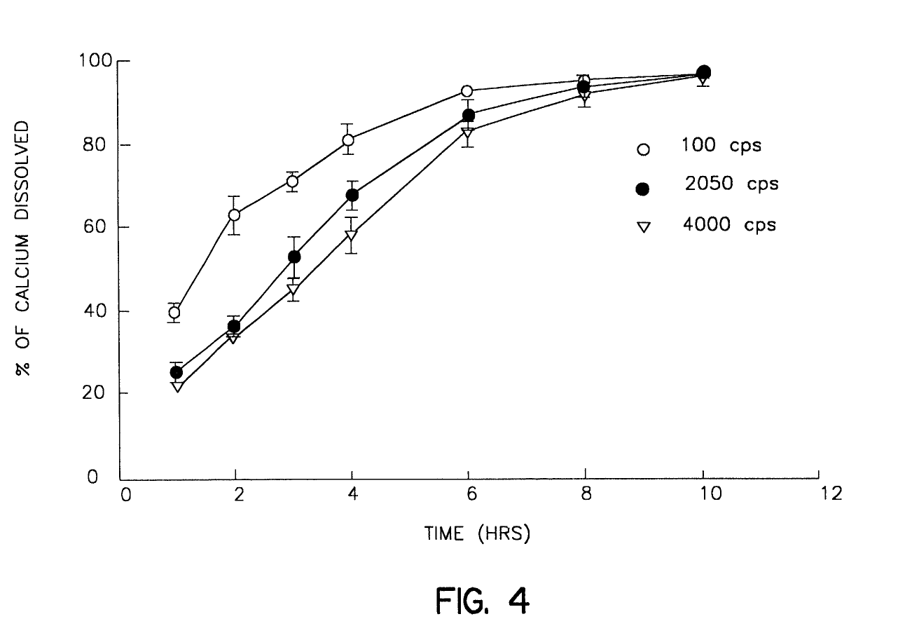

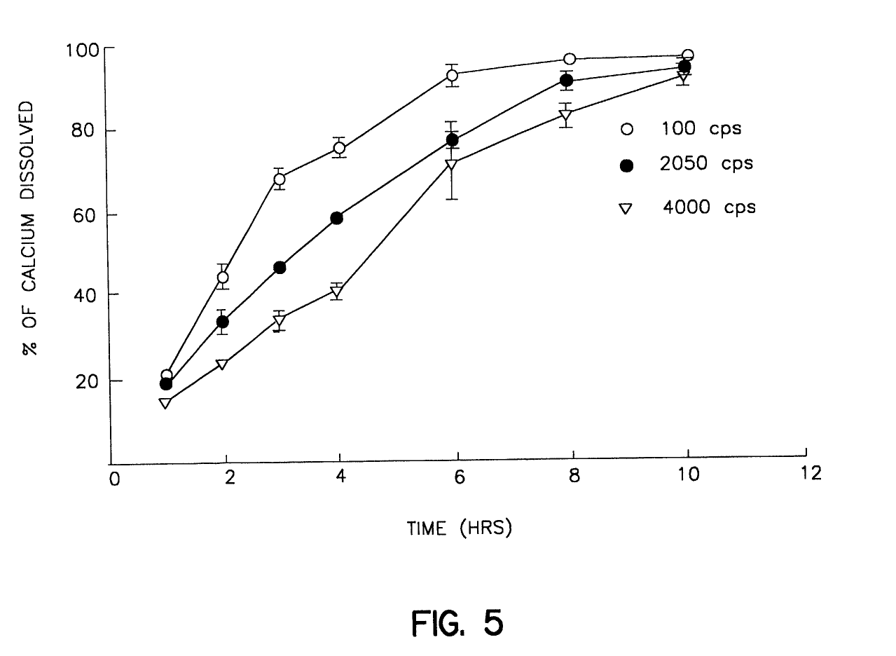

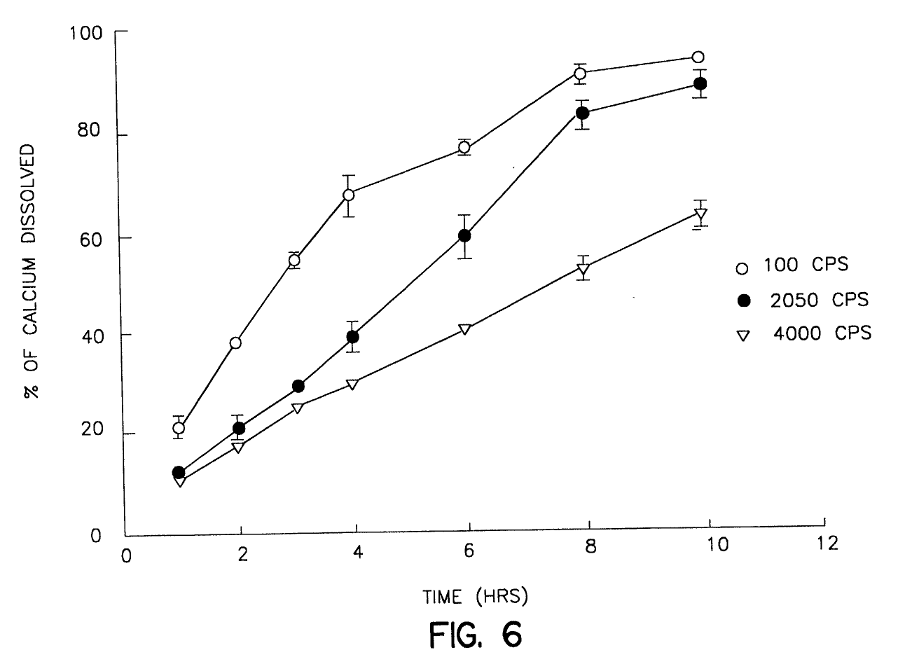

The effect of viscosity of the polymeric systems is illustrated in FIGS. 1, 2, and 3, and the effect of concentration is illustrated in FIGS. 4, 5 and 6. It can be seen that there is a fairly wide range of dissolution among the nine formulations. There are only two formulations that average over 90% at 6 hours. 5% K100LV (100 cps) has a dissolution of 92.7±1.42 (mean±1 SD, n=4) while 12.5% K100LV, 100 cps has a dissolution of 91.8% ±2.83.

Methocel K4M, 20%, (4000 cps) gave the lowest dissolution, releasing only about 40.7% of the calcium by 6 hours. All other formulations had an intermediate dissolution ranging from 58.8% to 86.6%. It can also be seen that in all formulations an inverse relationship exists between concentration of the HPMC matrix, viscosity of the HPMC matrix and calcium release rate. The higher the concentration and viscosity, the lower the calcium release rate.

### Example 2

A tablet consisting of ingredients, described in table 3 below, may be prepared using standard methods well recognized to those skilled in the art. The dissolution of the tablets of this example may be conducted in a manner similar to that defined above and shown in Tables 1 and 2.

TABLE 3

| Ingredient | Amount | Percent |
|---|---|---|
| Alginic acid | 260 mg | 26.0 |
| Sodium alginate | 260 mg | 26.0 |
| Sodium bicarbonate | 225 mg | 22.5 |
| Calcium carbonate | 160 mg | 16.0 |
| Sodium hydroxide | 30 mg | 3.0 |
| Carbomer | 65 mg | 6.5 |
| Preservative and flavor | q.s. | q.s. |

### Example 3

A tablet consisting of ingredients, described in table 4 below, may be prepared using standard methods well recognized to those skilled in the art. The dissolution of the tablets of this example may be conducted in a manner similar to that defined above and shown in Tables 1 and 2.

TABLE 4

| Ingredient | Amount | Percent |
|---|---|---|
| Alginic acid | 260 mg | 26.0 |
| Sodium alginate | 260 mg | 26.0 |
| Calcium carbonate | 160 mg | 16.0 |
| Preservative and flavor | q.s. | q.s. |

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the are can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A pharmaceutically acceptable oral calcium supplement preparation comprising a source of calcium ion in solid dosage form wherein the calcium source is entrapped with a hydrophilic matrix polymer, which polymer is a rafting agent, which polymer increases the residence time in the GI tract of the calcium supplement.

2. A preparation as claimed in claim 1 wherein the calcium source is calcium carbonate.

3. A preparation as claimed in claim 1 wherein the rafting agent is alginic acid and salts thereof.

4. A preparation as claimed in claim 3 which further comprises a second hydrophilic matrix polymer.

5. A preparation as claimed in claim 4 wherein the second hydrophilic matrix polymer is hydroxypropylmethylcellulose of varying molecular weight.

6. A preparation as claimed in claim 5 wherein the hydroxypropylmethylcellulose is composed of one or more different viscosity grades

7. A preparation as claimed in claim 5 wherein the viscosity grades of the hydroxypropylmethylcellulose are individually 100cps, 2500cps and 4000 cps.

8. A preparation as claimed in claim 5 wherein hydrophilic matrix polymer is hydroxypropylmethylcellulose of 100 cps.

9. A preparation as claimed in claim 5 wherein hydrophilic matrix polymer is hydroxypropylmethylcellulose of 2500 cps.

10. A preparation as claimed in claim 5 wherein hydrophilic matrix polymer is hydroxypropylmethylcellulose of 4000 cps.

11. A method of increasing calcium absorption in a mammal in need thereof, which method comprises administering to said mammal, a pharmaceutically acceptable oral preparation containing a source of calcium ion in solid form encapsulated with a hydrophilic matrix polymer, which is a rafting agent, whereby the polymer increases the residence time in the GI tract of the calcium supplement allowing increased absorption of the calcium.

12. A method as claimed in claim 1 wherein the calcium source is calcium carbonate.

13. A method as claimed in claim 11 wherein the rafting agent is alginic acid and salts thereof.

14. A method as claimed in claim 13 wherein the oral preparation further comprises a second hydrophilic matrix polymer.

15. A method as claimed in claim 15 wherein the second hydrophilic polymer is hydroxypropylmethylcellulose of varying molecular weight.

16. A method as claimed in claim 15 wherein the hydroxypropylmethylcellulose is composed of one or more different viscosity grades.

17. A method as claimed in claim 16 wherein the viscosity grades are 100cps, 2500cps and 4000 cps.

18. A method as claimed in claim 16 wherein the viscosity grade is 100cps.

19. A method as claimed in claim 16 wherein the viscosity grade is 2500cps.

20. A method as claimed in claim 16 wherein the viscosity grades is 4000 cps.

21. A process for increasing calcium absorption in a mammal in need thereof, which process comprises forming a matrix around a pharmaceutically acceptable solid calcium source wherein the matrix-forming material comprises hydroxypropyl methyl cellulose and a rafting agent.

22. The process according to claim 21 wherein the calcium ion source is calcium carbonate.

23. The process as claimed in claim 21 or 22 wherein the hydroxypropyl methyl cellulose has a viscosity grade of 100cps, 2500cps or 4000 cps.

24. The process as claimed in claim 23 wherein the rafting agent is alginic acid and salts thereof.
